(19) United States
(12) Patent Application Publication
(10) Pub. No.: US 2023/0032304 A1
(43) Pub. Date: Feb. 2, 2023

(54) SYSTEMS AND METHODS FOR QUANTIFYING HAZARDS IN LIVING SPACES

(71) Applicant: MatrixCare, Inc., Bloomington, MN (US)

(72) Inventors: Kedar Mangesh Kadam, Eden Prairie, MN (US); Michele N. Spadola, Carmel, NY (US); Kelly Keefe, Antioch, IL (US); Mary Beth Mohn, Bloomington, MN (US); Lee Eric Kilmer, Stillwater, MN (US); Adhiraj Ganpat Prajapati, St. Louis Park, MN (US)

(21) Appl. No.: 17/772,909

(22) PCT Filed: Oct. 30, 2020
(86) PCT No.: PCT/US2020/058085

§ 371 Date: Apr. 28, 2022

Related U.S. Application Data
(60) Provisional application No. 62/928,849, filed on Oct. 31, 2019.

(51) Int. Cl. G16H 50/30; G16H 40/60; G16H 15/00; G16H 10/60

(52) U.S. Cl. CPC G16H 50/30; G16H 40/60; G16H 15/00; G16H 10/60

(57) ABSTRACT

A system includes a sensor, a memory, and a control system. The sensor is configured to generate data associated with a living space of a resident. The memory is configured to store machine-readable instructions and a resident profile. The control system includes one or more processors configured to execute the machine-readable instructions to analyze the data to identify one or more hazards in the living space. The control system is further configured to execute the machine-readable instructions to determine a risk score for the living space based at least in part on the identified one or more hazards.

### CROSS-REFERENCE TO RELATED APPLICATIONS

This application claims the benefit of and priority to U.S. Provisional Patent Application No. 62/928,849, filed Oct. 31, 2019, which is hereby incorporated by reference herein in its entirety.

### TECHNICAL FIELD

The present disclosure relates generally to systems and methods for quantifying hazards in a living space, and more particularly, to systems and methods for determining a presence of hazards in the living space and providing a score for how dangerous the living space might be for a specific individual.

### BACKGROUND

Part of the human condition involves generally falling ill or becoming injured, which is why healthcare has been a big part of civilization for countless generations. Healthcare has evolved from simple rules and patterns (e.g., improving hygiene to avoid needlessly getting sick, eating unbruised fruits and vegetables, eating certain types of meals to reduce digestive effort, etc.) to more sophisticated methods involving curated plans by nutritionists, doctors, physical therapists, etc. As healthcare continues to evolve toward more reliance on experts, many individuals may be left behind due to several factors. For example, individuals may not have health insurance that covers a certain aspect of their health treatment, individuals may live in remote areas with a dearth of experts compared to more populated areas, individuals may relegate certain types of recommended care as insubstantial based on their health history, etc. Increasing access to expert recommendations and making these recommendations more affordable can generally improve healthcare in society.

Healthcare has grown to be a comprehensive term that not only encompasses diagnosis and treatment of injuries, illnesses, and diseases, but also to include illness and injury preventative measures. Preventative measures can sometimes take a backseat to diagnosis and treatment due to society's focus on the latter. As such, compliance with preventative measures can be much lower especially when an individual feels great. The present disclosure is directed to solving problems related to implementation of preventative measures and other problems.

### SUMMARY

According to some implementations of the present disclosure, a method includes receiving data associated with a living space of a resident. The received data is analyzed to identify one or more hazards in the living space. A risk score is determined for the living space based at least in part on the identified one or more hazards.

According to some implementations of the present disclosure, a system includes a sensor, a memory, and a control system. The sensor is configured to generate data associated with a living space of a resident. The memory is configured to store machine-readable instructions and a resident profile. The control system includes one or more processors configured to execute the machine-readable instructions to analyze the data to identify one or more hazards in the living space. The control system is further configured to execute the machine-readable instructions to determine a risk score for the living space based at least in part on the identified one or more hazards.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a perspective view showing an individual scanning a living area, according to some implementations of the present disclosure;

FIG. 2 is a block diagram of a system for determining hazards in a living area, according to some implementations of the present disclosure;

FIG. 3 is a flow diagram illustrating steps for determining hazards in a living space and rating the living space based on the determined hazards, according to some implementations of the present disclosure;

FIG. 4 is a flow diagram illustrating steps for a user device to provide recommendations for reducing hazards in a living space, according to some implementations of the present disclosure;

FIG. 5 is a flow diagram illustrating steps for refreshing recommendations based on an updated risk score, according to some implementations of the present disclosure;

FIG. 6A illustrates a mobile device with the screen of the mobile device displaying a living space, according to some implementations of the present disclosure;

FIG. 6B illustrates the screen of the mobile device of FIG. 6A displaying a change in furniture arrangement, according to some implementations of the present disclosure;

FIG. 7A illustrates a mobile device with the screen of the mobile device displaying a bathroom, according to some implementations of the present disclosure; and

FIG. 7B illustrates the mobile device of FIG. 7A with the screen displaying grab bars in the bathroom, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Individuals are known to spend considerable amount of time at their homes. An individual may frequent certain spaces in her home when compared to other spaces. For example, the individual may spend more time in her bedroom and less time in her living room. Overall, based solely on how much time the individual spends in her bedroom and her living room, there is a higher chance that an injury to the individual would take place in her bedroom than in her living room. Some implementations of the present disclosure provide systems and methods to reduce risk of injury or to reduce potential hazards in spaces where the individual spends her time.

Reducing risk of injury or reducing potential hazards can be important in improving or maintaining an individual's quality of health. Reducing risk of injury falls under preventative healthcare and can reduce hospitalization and/or re-hospitalization. For example, a person has a health condition that prompts a visit to a facility or a hospital. When the person is discharged to go home from the facility, the health condition or treatment administered at the facility can place the person under an increased risk of injury and/or re-injury. For example, a person who breaks an arm or a leg can re-injure the arm or the leg from unexpectedly slipping and falling, even though the fall itself may not have caused injury if she were healthy.

The present disclosure provides systems and methods for addressing hazards in a living space. The hazards can be hazardous to one or more residents and/or individuals (and/or pets) in the living space (e.g., home). Such resident(s) can be elderly (e.g., over the age of 50, 60, 70, 80, 85, 90, 95, 100, etc.), developmentally challenged, cognitively challenged, handicapped, physically limited, etc., or any combination thereof.

Some implementations of the present disclosure provide systems and methods for scanning a living space of individuals who are at higher risks of being re-injured to identify hazards within the living space. Conventional methods of auditing a living space to reduce possibility of adverse events that can lead to re-hospitalization can be time consuming and expensive. Usually, a qualified and/or trained individual affiliated with the hospital visits the living space to make recommendations of how the living space should be adjusted. Some implementations of the present disclosure eliminate the need for having the qualified and/or trained individual or expert scan the living space.

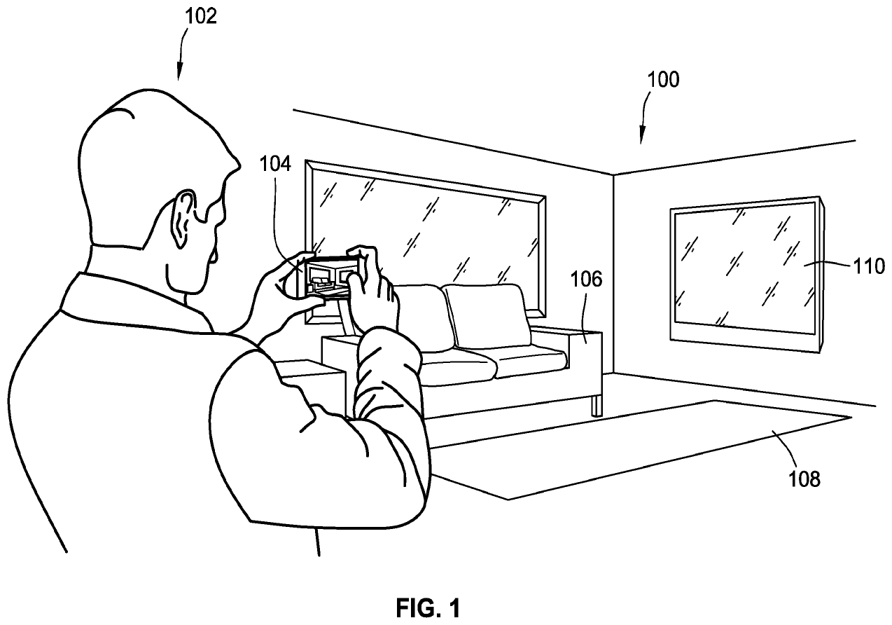

Referring to FIG. 1, an individual 102 is scanning a living area, according to some implementations of the present disclosure. The individual 102 is using a smartphone 104 to scan a living room 100. The living room 100 includes a couch 106, a rug 108, a wall-mounted television 110, etc. The individual 102 scans the living room 100 by taking videos and/or photos of the living room 100. The individual 102 does not have to be an expert in home healthcare, but can rely on software running, for example, on the smartphone 104 to provide recommendations on which aspects of the living room 100 should be adjusted to make the living room safer for a resident (e.g., by removing one or more hazards or potential hazards to the resident). By relying on the software, auditing a living space according to some implementations of the present disclosure can be performed relatively faster and at a lower cost compared to conventional methods. Further, an expert in home healthcare is not required to audit the living space. Rather, a family member or friend of the resident is able to conduct the audit using the software of the present disclosure (e.g., running on the smartphone 104).

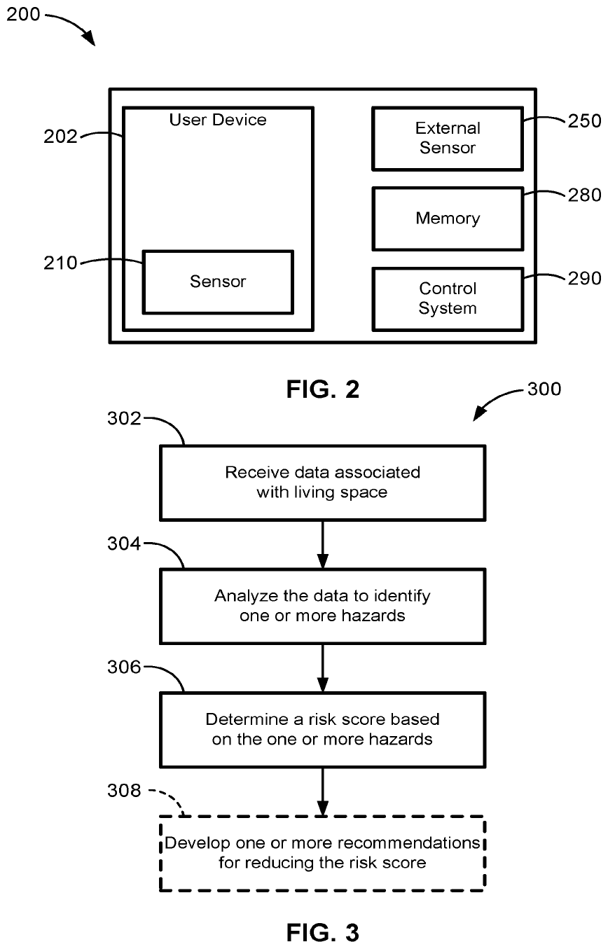

Referring to FIG. 2, a system 200 includes a user device 202 with a sensor 210, a control system 290, and a memory 280. In some implementations, the system 200 also includes an external sensor 250. To simplify discussion, the singular form will be used for all components identified in FIG. 2 when appropriate, but the use of the singular does not limit the discussion to only one of each component. The system 200 generally can be used to generate data associated with a living space (e.g., living room 100 shown in FIG. 1) of a resident and analyze the generated data to determine and/or identify one or more hazards within the living space of the resident. In some implementations, the system 200 also provides recommendations to reduce one or more of the determined and/or identified hazards within the living space.

The control system 290 includes one or more processors. As such, the control system 290 can include any suitable number of processors (e.g., one processor, two processors, five processors, ten processors, etc.). In some implementations, the control system 290 includes one or more processors, one or more memory devices (e.g., the memory 280, or a different memory device), one or more other electronic components (e.g., one or more electronic chips/components, one or more printed circuit boards, one or more power units, one or more graphical processing units, one or more input devices, one or more output devices, one or more secondary storage devices, one or more primary storage devices, etc.), or any combination thereof. In some implementations, the control system 290 includes the memory 280 or a different memory device, yet in other implementations, the memory 280 is separate and distinct from the control system 290, but in communication with the control system 290.

The control system 290 generally controls (e.g., actuate) the various components of the system 200. In some implementations, the control system 290 analyzes data obtained and/or generated by the components of the system 200 and/or received from other components not included in the system 200. For example, the control system 290 is arranged to provide control signals to the external sensor 250. The control system 290 executes machine-readable instructions that are stored in the memory 280 or a different memory device. The control system 290 can implement one or more engines of the system 200. An engine is a combination of hardware and software configured to perform specific functionality. The one or more processors of the control system 290 can be general or special purpose processors and/or microprocessors.

While the control system 290 is described and depicted in FIG. 2 as being a separate and distinct component of the system 200, in some implementations, the control system 290 is integrated in and/or directly coupled to the user device 202 and/or the external sensor 250. The control system 290 can be coupled to and/or positioned within a housing of the user device 202, the external sensor 250, or any combination thereof. The control system 290 can be centralized (within one housing) or decentralized (within two or more physically distinct housings, e.g., having a first portion in a housing of the user device and a second portion in a separate housing).

While the system 200 is shown as including a single memory 280, it is contemplated that the system 200 can include any suitable number of memory devices (e.g., one memory device, two memory devices, five memory devices, ten memory devices, etc.). The memory 280 can be any suitable computer readable storage device or media, such as, for example, a random or serial access memory device, a hard drive, a solid-state drive, a flash memory device, etc. The memory 280 can be coupled to and/or positioned within a housing of the user device 202 and/or the external sensor 250. The memory 280 can be centralized (within one housing) or decentralized (within two or more physically distinct housings).

The user device 202 can include a mobile phone, a personal digital assistant, a tablet, a laptop computer, a handheld camera, or any combination thereof. The sensor 210 and/or the external sensor 250 can include a camera, an infrared camera, a radio-frequency (RF) sensor, an ultra-wide band RF sensor, an infrared sensor, an acoustic sensor, a radar sensor, a heat sensor, a gas sensor, a microphone, a speaker, a barometer, a proximity sensor, a humidity sensor, a gyroscopic sensor, a light sensor, a motion sensor, a Light Detection and Ranging (LiDAR) sensor, or any combination thereof.

In some implementations, the sensor 210 and/or the external sensor 250 includes a camera that generates and/or outputs image data reproducible as one or more images (e.g., still images, video images, or both) that can be stored in the memory 280 and/or one or more other memory devices. The image data from the camera can be used by the control system 290 (and/or the user device 202) to map or digitally represent a living area for use in determining one or more hazards within in the mapped living area.

In some implementations, the sensor 210 and/or the external sensor 250 includes an acoustic sensor, and the acoustic sensor can include a microphone and a speaker. The speaker generates or emits sound waves at a predetermined interval and the microphone detects the reflections of the emitted sound waves from the speaker. The sound waves generated or emitted by the speaker have a frequency that is not audible to the human ear (e.g., below 20 Hz or above about 18 kHz). Based at least in part on the data from the microphone and the speaker, the control system 290 can determine a mapping or a digital representation of a living area and/or one or more portions of the living area and/or one or more items in the living area.

In some implementations, the sensor 210 and/or the external sensor 250 includes an RF sensor, and the RF sensor can include an RF receiver and an RF transmitter. The RF transmitter generates and/or emits radio waves having: (i) a predetermined frequency, (ii) a predetermined amplitude (e.g., within a high frequency band, within a low frequency band, long wave signals, short wave signals, etc.), (iii) continuous waves (e.g., CW, FMCW), (iv) pulsed waves (e.g., pulsed CW, UWB, etc.), (v) coded waves (e.g., PSK, FSK, etc.), or (vi) any combination thereof. The RF receiver detects the reflections of the radio waves emitted from the RF transmitter, and the detected reflections are output by the RF receiver as data that can be analyzed by the control system 290 to determine a mapping or a digital representation of a living area and/or one or more portions of the living area and/or one or more items in the living area. The RF receiver and/or the RF transmitter can also be used for wireless communication between the control system 290, the user device 290, the external sensor 250, or any combination thereof. In some implementations, the RF sensor can include a transceiver that acts as both the RF receiver and the RF transmitter.

In some implementations, the sensor 210 and/or the external sensor 250 includes an infrared (IR) sensor or a heat sensor that generates and outputs infrared image data reproducible as one or more infrared images (e.g., still images, video images, or both) that can be stored in the memory 280 and/or one or more other memory devices. The IR sensor can also be used in conjunction with a camera when measuring movement of the resident. The IR sensor can detect infrared light having a wavelength between about 700 nm and about 1 mm, while the camera can detect visible light having a wavelength between about 380 nm and about 740 nm. The control system 290 can distinguish warm blooded pets in a living area using the image data generated from the IR sensor and the camera.

In some implementations, the sensor 210 and/or the external sensor 250 includes a LiDAR sensor for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor can measure and map an area extending 5 meters or more away from the sensor. The LiDAR data can be fused with point cloud data estimated by an electromagnetic RADAR sensor, for example. The LiDAR sensor can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the sensor 210 and/or the external sensor 250 includes a moisture sensor that outputs data that can be stored in the memory device 280 and used by the control system 290. The moisture sensor can be used to detect moisture in various areas surrounding the individual 102. The moisture sensor can be placed near any area where moisture levels need to be monitored or integrated in the user device 202. The moisture sensor can also be used to monitor the humidity of the ambient environment surrounding the individual 102, for example, the air inside the bedroom.

In some implementations, the sensor 210 and/or the external sensor 250 includes a gas sensor that outputs data related to air quality of the ambient environment surrounding the individual 102. For example, the gas sensor can be used to determine whether there is a gas leak from a gas line used for cooking and/or heating. The gas sensor can be used to determine whether there is an excess of carbon-dioxide, carbon-monoxide, methane, etc., in the ambient environment surrounding the individual 102. The gas sensor can be used to determine levels of nitrogen, oxygen, or any other type of gas. Air quality within the ambient environment surrounding the individual 102 can indicate whether the air of the living space is toxic or unsafe for a resident.

The sensor 210 and/or the external sensor 250 can generate sensor data that is associated with and/or reproducible as a living space of a resident for use in quantifying one or more hazards within the living space. Referring to FIG. 3, a method 300 for quantifying a hazardous nature of a living space by the system 200 is illustrated. At step 302, the user device 202 (and/or the control system 290) receives data associated with the living space. Data associated with the living space can include data generated from the sensor 210 and/or the external sensor 250. Such received data can include image data from a camera, data from an infrared sensor, image data from an IR sensor and/or a heat sensor, data from an acoustic sensor, data from a radar sensor, data from an ultra-wide band RF sensor, data from a microphone, data from a speaker, data from an RF sensor, data from a gas sensor, data from a barometer, data from a proximity sensor, data from a humidity sensor, data from a gyroscopic sensor, data from a light sensor, data from a motion sensor, or any combination thereof.

Data associated with the living space can include user inputs received via the user device 202 and/or data from the memory 280. For example, a user scanning the living space (e.g., using the user device 202) can use input devices on the user device 202 to set parameters associated with the living space and/or the user device 202 can obtain the parameters associated with the living space from a remote database storing a resident profile. In some implementations, the resident profile includes an electronic health record associated with the resident. In some implementations, the resident profile includes parameters that define a height of a resident, an age of the resident, a health condition from which the resident is currently recovering from, a health condition from which the resident is currently enduring, whether the resident has a roommate or is living alone, a type of home of the resident, medication prescribed to the resident, vital signs of the resident, a type of assistive device used by the resident, a mental status of the resident, demographic information of the resident, a location of a bedroom of the resident within the living space, a location of a bathroom of the resident within the living space, an average number of times the resident uses the bathroom per day in the living space, an average activity level of the resident per day, or any combination thereof.

A type of home/residence of the resident can include whether the resident lives in an apartment complex, a single-family home in a subdivision, a number of floors within the living space, etc. A type of assistive device used by the resident can include a wheelchair, a walker, etc. Demographic information of the resident can include an age of the resident, an address of the resident, details on a care team of the resident (e.g., doctors, nurses, emergency contact, etc.), previous occupation, current occupation, or any combination thereof.

At step 304, the user device 202 and/or the control system 290 analyzes the data received at step 302 to identify one or more hazards in the living space. In some implementations, the user device 202 and/or the control system 290 determines that one or more physical objects are contained within the living space. For example, the user device 202 and/or the control system 290 can use the received data to recognize that the living space includes one or more doorways, one or more pieces of furniture within the living space, one or more carpeted floors, one or more light fixtures, one or more hardwood floors, one or more electrical outlets, one or more stairs, one or more appliances, one or more cabinets, one or more pieces of clothing, one or more shoes, one or more beds, one or more pets, etc. The user device 202 can analyze video data and/or image data from the sensor 210 and/or the external sensor 250 to identify the one or more physical objects contained within the living space.

In some implementations, the user device 202 and/or the control system 290 determines and/or generates and/or creates a digital map of the living space. The digital map of the living space is a computational representation of the living space such that the user device 202 and/or the control system 290 can recognize that the living space includes one or more walkways, one or more physical objects at specific locations within the living space, etc. Examples of computational representations include multi-dimensional matrices and transformations of such matrices to describe three-dimensional space and/or two-dimensional space. The user device 202 and/or the control system 290 can link each recognized physical object to a specific location within the digital map. For example, the user device 202 and/or the control system 290 can determine that a sink is located within a bathroom of the living space. The user device 202 can use data contained in a resident profile to identify a bathroom of the resident within the digital map and to indicate that certain physical objects are located within the bathroom of the resident. Discussion of the bathroom and physical objects in the bathroom are used as an example. The user device 202 can leverage data from the sensor 210, the external sensor 250, and/or the resident profile to contextualize where different physical objects are located within the living space.

In some implementations, the user device 202 and/or the control system 290 can add further context surrounding the physical objects. The user device 202 and/or the control system 290 can determine sizes of the physical objects within the living space using the received data from step 302. For example, the sensor 210 can include a single camera, and the user device 202 and/or the control system 290 can use one or more image processing algorithms on image data captured by the single camera to determine a width of one or more physical walkways within the living space, a distance between physical objects within the living space, sizes of physical objects within the living space, one or more dimensions of physical objects within the living space, or any combination thereof. In another example, the sensor 210 can include two separate and distinct cameras for determining sizes of the physical objects within the living space. A distance separating the two separate and distinct cameras can be used as a reference distance to determine, from image data collected by the two separate and distinct cameras, a width of one or more physical walkways within the living space, a distance between physical objects within the living space, sizes of physical objects within the living space, one or more dimensions of physical objects within the living space, or any combination thereof.

In some implementations, the user device 202 and/or the control system 290 can identify features within the digital map. For example, within the digital map, the user device 202 and/or the control system 290 can identify the one or more hazards from a hazards database, the identified physical objects, the identified physical walkways, specific locations of the identified physical objects, specific locations of the identified physical walkways, or any combination thereof. The hazards database can be included in the memory 280. The hazards database stores potential hazards that can be found in living spaces. For example, the hazards database can include that one or more obstacles blocking a walkway in a living space is a hazard, one or more loose cords in a living space is a hazard, etc. The user device 202 can perform a matching between hazards found within the hazards database and the identified physical objects, the identified walkways, and/or the specific locations of the identified physical objects and walkways.

Examples of the one or more hazards include one or more obstacles blocking a walkway of the living space, a narrow walkway of the living space, one or more tripping hazards in the living space (e.g., an uneven threshold, a step, a rug, etc.), one or more loose cords in the living space, one or more loose wires in the living space, a slippery floor of the living space, a relatively hard to reach light switch in the living space, a relatively hard to reach electrical outlet in the living space, a low toilet seat in the living space, a lack of shower handles in a shower in the living space, a sharp corner in the living space, a sharp edge in the living space, one or more pets in the living space, one or more stairs in the living space, a gas stove in the living space, a high positioning of cabinets in the living space, a difficult-to-use electrical appliance in the living space, a height of a bed in the living space, a height of a sink in a bathroom of the living space, a dimly lit area in the living space, a type of shower or tub of the living space, or any combination thereof.

At step 306, the user device 202 and/or the control system 290 determines a risk score for the living space based at least in part on the identified one or more hazards of step 304. The risk score for the living space can be indicative of how hazardous the living space is to the resident. In some implementations, the risk score can more generally be indicative as to how hazardous the living space would be to anyone, not just the resident (e.g., a guest of the resident). As such, in some implementations, the user device 202 and/or the control system 290 can use information from the resident profile to determine the risk score.

In some implementations, the user device 202 and/or the control system 290 determines the risk score for the living space by determining a hazard score for each of the one or more identified hazards in the living space. For each of the one or more identified hazards, the user device 202 and/or the control system 290 can obtain a respective hazard score from a database (e.g., the hazards database). The hazards database can include a chart or look-up table with predefined hazard scores for each of the one or more identified hazards. The user device 202 and/or the control system 290 can then combine the hazard scores to determine the risk score of the living space.

In some implementations, the one or more hazards are linked to potential harms that can befall the resident. For example, loose cords are a tripping hazard that can potentially cause the resident to fall and injure herself. The user device 202 and/or the control system 290 can determine likelihood of occurrences for potential harms linked to the one or more hazards. The user device 202 and/or the control system 290 can take into account a health condition of the resident when determining the likelihood of occurrences for the potential harms. For example, a relatively hard to reach light switch in the living space can be deemed an actual hazard for some residents because a resident in a wheelchair trying to reach a switch that is blocked by a kitchen counter will have a hard time and can possibly harm themselves in exerting effort to reach the switch. Whereas a resident not in a wheelchair will not have a similar problem. As such, a likelihood of occurrence of a potential harm related to a relatively hard to reach light switch is very low for a non-wheelchair bound resident compared to the resident in the wheelchair.

In some implementations, the user device 202 and/or the control system 290 determines a likelihood of occurrence associated with the one or more hazards of step 304. The likelihood of occurrence associated with a hazard can be determined based on a frequency of the resident's interaction with the one or more hazards. For example, a personal bathroom of a resident in a wheelchair includes grab bars to help prevent falls in the shower. A guest bathroom of the resident in the wheelchair on the other hand does not include grab bars, but since the resident does not use the guest bathroom, the frequency of the resident's interaction with physical objects within the bathroom is very low. As such, a likelihood of occurrence associated with hazards found within the guest bathroom is relatively lower while a likelihood of occurrence associated with hazards found within the personal bathroom of the resident is relative higher.

In some implementations, the likelihood of occurrence associated with the one or more hazards of step 304 can be expressed as a percentage likelihood for each of the one or more hazards of step 304. The user device 202 and/or the control system 290 can determine the percentage likelihood(s) based at least in part on a historical frequency of proximity of the resident to the one or more hazards. Data associated with a movement of the resident throughout the living space can be generated by the external sensor 250. The movement of the resident throughout the living space can include locations within the living space that the resident frequents. The control system 290 can determine from the movement of the resident the historical frequency of proximity of the resident which includes whether the resident settles more often in certain areas within the living space or whether the resident interacts more often with certain hazards within the living space. The historical frequency of proximity can include data in three-day intervals, one week intervals, one month intervals, etc.

In some implementations, since the resident's pattern can change over time, the control system 290 can use a recency biasing on the historical frequency of proximity when determining the likelihood of occurrence associated with the one or more hazards based on the historical frequency of proximity. That is, more recent interactions and/or positions of the resident in the living space(s) can be weighed more heavily.

In some implementations, a magnitude of the likelihood of occurrence for a respective one of the one or more hazards is directly proportional to the historical frequency of proximity. That is, a first hazard with more historical interactions by the resident will have a higher magnitude for the likelihood of occurrence compared to a second hazard with less historical interactions by the resident.

Examples are provided where likelihood of occurrence can be harm-dependent or can be based on a historical frequency of proximity. Other factors can also affect likelihood of occurrence of the one or more hazards. For example, additional information from a resident profile can be taken into account when determining likelihood of occurrence. The demographic information of the individual can be taken into account. For example, if stairs are identified as a hazard in respective living spaces for a number of residents, then likelihood of occurrence associated with the stairs can be organized from highest to lowest as follows (taking into account circumstances surrounding the resident): (1) highest for an eighty-year-old recovering from hip replacement surgery, (2) higher for a healthy eighty-year-old without an alternative means like an elevator, (3) high for a healthy eighty-year-old with an alternative means that bypasses the stairs, and (4) lowest for a healthy thirty year old without an alternative means that bypasses the stairs.

In some implementations, the user device 202 and/or the control system 290 determines the risk score via a weighted average of a hazard score of each of the one or more identified hazards and a corresponding likelihood of occurrence. For example, the user device 202 and/or the control system 290 weights the hazard score for each of the one or more identified hazards by a corresponding likelihood of occurrence and adds each of the weighted hazard scores to obtain the risk score. A weighted average can take into account that a hazard being identified in the living space does not mean that the hazard carries a same risk factor(s) as other hazards. In the example above with the personal bathroom and the guest bathroom, if the resident does not frequent the guest bathroom and/or rarely comes into contact with the identified hazards, then the likelihood of occurrence associated with hazards within the guest bathroom will be relatively lower compared to the likelihood of occurrence associated with hazards within the personal bathroom. Determining the risk score with the weighted average including the likelihood of occurrence can reduce contribution of hazard scores associated with the identified hazards within the guest bathroom. As such, the risk score calculated can be more tailored to a specific resident and can more accurately represent an aggregate risk measure of the living space to the specific resident's use of the living space (as compared with some other person with different challenges that is not a resident of the living space).

In some implementations, a first living space for a resident with a first calculated risk score that is greater than a second calculated risk score for a second living space for the resident indicates that the first living space is more hazardous to the resident than the second living space. Demographic information and other circumstantial information can be incorporated into the risk score by determining likelihood of occurrences and using the likelihood of occurrences to scale (or weight) hazard scores associated with one or more hazards identified in a living space. Incorporating likelihood of occurrences in the risk score allows tailoring the risk score to convey more meaningful information to specific residents.

Optionally, at step 308, the user device 202 and/or the control system 290 develops one or more recommendations for reducing the risk score determined at step 306. The one or more recommendations for reducing the risk score include removing the hazard, making one or more modifications to the living space, moving one or more items in the living space, add one or more items to the living space, or any combination thereof. For example, the recommendations can include a recommendation to increase a width of a physical walkway of the living space, remove a first physical object from the living space, reorient a second physical object within the living space, add one or more new objects to the living space, or any combination thereof. For another example, a resident recently provided with a wheelchair may need walkways and/or doorways in her apartment to be resized for permitting passage through the walkways and/or doorways. In another example, the user device 202 and/or the control system 290 can recommend that a rug be removed from the living space since the rug may interfere with operating a wheelchair to access areas within a living space. The one or more recommendations can be sent to a mobile or electronic device of a caregiver (e.g., a family member of the resident, a physician treating the resident, a guardian of the resident, anyone involved in medical care of the resident, etc.), a mobile device or electronic device of the resident, etc. The one or more recommendations can be printed via a printer and/or displayed on a screen of an electronic device (e.g., the smartphone 104).

In some implementations, the one or more recommendations are based on suggestions included in a hazards database. For example, the hazards database may not only include hazards but can also include several methods of mitigating risks associated with the hazards. The user device 202 and/or the control system 290 can pull, from the hazards database, the one or more recommendations based on the one or more hazards identified at step 304.

In some implementations, the one or more recommendations are ranked based on a potential impact to the risk score. For example, if a first hazard contributes more to the risk score than a second hazard, then a recommendation to reduce or eliminate the first hazard is ranked as being relatively more important than a recommendation to reduce or eliminate the second hazard. In some implementations where the risk score is determined primarily on predefined hazard scores as discussed above with respect to step 304, then the one or more recommendations are ranked based on an ascending or descending order of predefined hazard scores of the identified one or more hazards. In some implementations where the risk score takes into account likelihood of occurrence as discussed above with respect to step 306, the one or more recommendations are ranked based on an ascending or descending order of hazard scores weighted with corresponding likelihoods of occurrence associated with the hazard scores. As such, the one or more recommendations can be tailored to the living space for each specific resident. For example, a recommendation to remove a rug can have a bigger impact for a resident in a wheelchair than a resident with a broken arm. Therefore, the recommendation to remove the rug would likely be ranked higher for the resident with the wheelchair as compared with the resident with a broken arm.

In some implementation of the present disclosure, each or some of the one or more recommendations are associated with and/or at least partially based on an available space in the living area for implementation of the recommendation(s). For example, if the recommendation is to add one or more grab bars in a bathroom (e.g., in the shower), the size and/or number of the one or more grab bars will be based at least in part on the space available in the bathroom.

In some implementations of the present disclosure, each or some of the one or more recommendations are associated with an estimated implementation cost. The user device 202 and/or the control system 290 can retrieve estimated costs for implementing the one or more recommendations from a database (e.g., the hazards database). Each recommendation in the hazards database can include an associated cost or price. For example, moving a piece of furniture or moving an object from one location to another may include a cost of $0, but adding grab bars in a bathroom of a resident can include an average price for a grab bar, an average price for installation tools, an average price for labor costs in a local area of the resident, or any combination thereof. In some implementations, an average price for labor costs are not included but only an average price of the item being recommended. In some implementations, a specific item from an online store is recommended with a specific price. The hazards database can be linked to several other databases to pull local price data for recommended items, determine average price data for recommended items, and/or determine average or specific labor costs for installing recommended items and/or removing non-recommended items.

In some implementations, the estimated implementation cost associated with the one or more recommendations takes into account methods of payment. For example, insurance information can be provided in the resident profile, and the user device 202 and/or the control system 290 can use the insurance information to determine covered and uncovered expenses.

That way, an estimated implementation cost provided can be adjusted so only out of pocket expenditures for the resident are provided.

In some implementations, the one or more recommendations are ranked based on an estimated cost. Recommendations with a more expensive price tag can be on the bottom of the list while recommendations with a less expensive price tag can be at the top. In some implementations, the one or more recommendations are ranked based on a comparison between a potential impact of a respective recommendation on the risk score and an estimated cost of implementing the respective recommendation. The potential impact can be determined according to some implementations of the present disclosure as described above. Ranking the comparison between the potential impact of the respective recommendation on the risk score and the estimated cost of implementing the respective recommendation includes: (i) ranking the recommendations in descending order based on a ratio of the potential impact of the respective recommendation on the risk score to the estimated cost of implementing the respective recommendation, or (ii) ranking the recommendations in ascending order based on a ratio of the estimated cost of implementing the respective recommendation to the potential impact of the respective recommendation on the risk score. That way, cost per impact can be maximized.

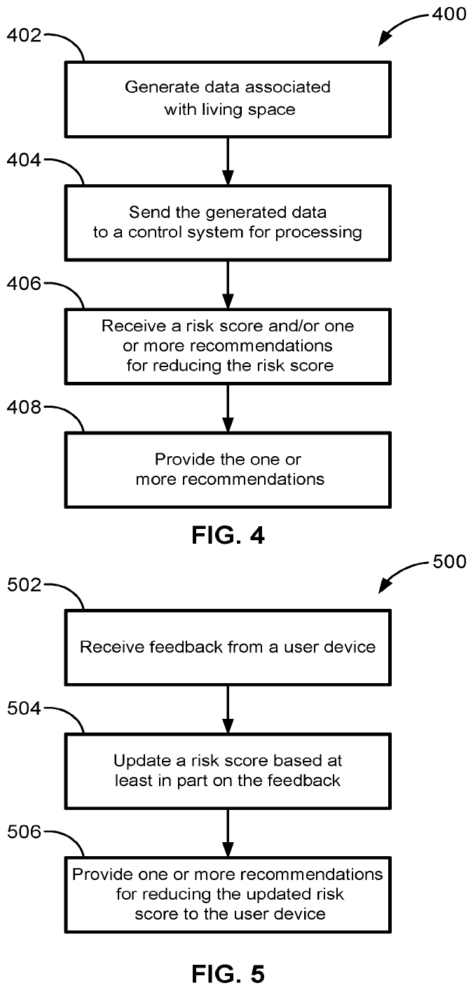

As described above, FIG. 3 is associated with a method that may be taken by the user device 202 and/or the control system 290. A more specific implementation involving an interaction between the user device 202 and the control system 290 is provided in FIG. 4. Referring to FIG. 4, a method 400 for providing a user of the user device 202 with recommendations for reducing hazards in a living space is illustrated. At step 402, the user device 202 generates data associated with the living space. Step 402 is similar to step 302 previously described in connection with FIG. 3.

At step 404, the user device 202 sends the generated data to the control system 290 for processing. The control system 290 can be a desktop computer, a remote computer, a server, a cloud service, etc. The control system 290 can include additional processing capabilities beyond that of the user device 202. The user device 202 offloads processing of the generated data of step 402 to the control system 290.

At step 406, the user device 202 receives a risk score and/or one or more recommendations for reducing the risk score. The risk score can be calculated according to the implementations of the present disclosure as described in connection with step 306 of FIG. 3. The one or more recommendations for reducing the risk score can be determined according to the implementations of the present disclosure as described in connection with step 308 of FIG. 3.

At step 408, the user device 202 presents and/or displays the one or more recommendations to the user in an understandable format, to the resident in an understandable format, to a caretaker of the resident in an understandable format, or a combination thereof. The user device 202 can display the one or more recommendations as a list of items in an email program and/or in some other application program. The user device 202 can send the one or more recommendations to a printer so that the printer can create physical paper copies with a list of items for reducing risk associated with identified hazards. The user device 202 can dictate the list of items to a user, the resident, a caretaker of the resident, etc. The user device 202 can provide a link to educational videos related to the one or more recommendations or can provide a video playlist that describes the one or more recommendations. The user device 202 can graphically display the one or more recommendations on a screen of the user device 202.

In some implementations, the user device 202 is configured to create a virtual representation of at least a portion of the living space of the resident via the generated data of step 402. The virtual representation of the at least a portion of the living space can include one or more virtual walkways, one or more virtual objects (e.g., rugs, pets, cords, lamps, televisions, pictures, switches, etc.), or any combination thereof. In some such implementations, the virtual walkway is a virtual representation of a physical walkway contained in the living space, and the virtual object is a virtual representation of a physical object contained in the living space. The virtual representation of the at least a portion of the living space can include one or more virtual items of furniture, one or more virtual rugs, one or more virtual carpeted floor areas, one or more virtual light fixtures, one or more virtual hardwood floor areas, one or more virtual electrical outlets, one or more virtual stairs, one or more virtual appliances, one or more virtual cabinets, one or more virtual items of clothing, one or more virtual shoes, a virtual bed, one or more virtual pets, one or more virtual doorways, one or more virtual walkways, or any combination thereof.

In some implementations, the user device 202 displays the one or more recommendations as one or more virtual objects within the virtual representation of the living space. The user device 202 can add virtual objects that do not currently have a physical object representation in the living space. For example, the user device 202 can display a cabinet lock on a cabinet even though the cabinet lock is not currently in the living space. The user device 202 can thus virtually augment the living space by adding one or more virtual objects. In some such implementations, the added virtual objects are added in response to one or more of the recommendations. The virtual objects that can be virtually added and/or overlaid on and/or in the virtual representation of the living space include, for example, a toilet seat lift, a bedside portable toilet, a shower chair, a grab bar, a static ramp, a movable lift, an adjustable lift chair to help the resident transition from sitting to standing, an adjustable bed like those provided in hospitals, a cabinet lock, a gas knob lock, a furniture strap to prevent furniture from falling over, a furniture bumper, a reacher or an extendible claw to extend a reach of the resident, a wheelchair, a walker, a cable management system, or any combination thereof.

An example of a movable lift is a chair lift that can be recommended for assisting the resident move up one or more stairs. The user device 202 can include a virtual chair lift alongside a virtual representation of stairs. The user device 202 can represent the added virtual objects from the one or more recommendations in many ways. For example, the user device 202 can use different icons on-screen as the virtual objects. A wheelchair can be represented with one icon, a chair lift can be represented with a different icon, etc. In some implementations, the different icons are photographs such that a photograph of a wheelchair is overlaid in the virtual representation of the living space or a photograph of a chair lift is overlaid in the virtual representation of the living space. In some implementations, the user device 202 renders a wheelchair with proportionate dimensions within the virtual representation of the living space or renders a chair lift with proportionate dimensions showing how the chair lift will fit alongside the stairs in the virtual representation of the living space. The virtual renderings of the chair lift or the wheelchair can be displayed in an augmented reality view of at least a portion of the living space being displayed on the screen of the user device 202. A wheelchair and a chair lift are used herein as examples, but other any other number of virtual objects can be virtually displayed in the virtual representation of the at least a portion of the living space.

In some implementations, the user device 202 can display one of the recommendations as an animation involving one or more moving virtual objects. For example, if a recommendation involves moving a furniture from one location to another, the user device 202 can show a virtual item of furniture changing locations. Other examples of moving recommendations include showing a virtual rug moving from one location to another, showing a virtual object moving from one location to another, etc.

In some implementations, the user device 202 can display the one of the recommendations as an animation involving a resizing of virtual objects or virtual walkways. For example, the animation can include a widening of a virtual walkway in the virtual representation of the living space, a narrowing of a virtual walkway in the virtual representation of the living space, a resizing of a virtual object in the virtual representation of the living space, a widening of a virtual doorway in the virtual representation of the living space, a narrowing of the virtual doorway in the virtual representation of the living space, or any combination thereof.

Referring to FIG. 5, a method 500 for refreshing recommendations based on an updated risk score is illustrated. In some implementations, the user device 202 has processing power to perform the method 500. In some implementations, the user device 202 offloads processing or a portion of the processing to a cloud server (e.g., the control system 290). FIG. 5 is described with respect to the control system 290, however, any control system can be used to implement some or all of the method 500. At step 502, the control system 290 receives feedback from the user device 202. The feedback can include implementation feedback, that is, feedback associated with an implementation status for one or more recommendations for reducing a risk score according to some implementations of the present disclosure. The implementation status can be binary, indicating whether each of the one or more recommendations was implemented or not.

The feedback can include which recommendations a resident or a user finds appealing. For example, the user can indicate on the user device 202 that a subset of the one or more recommendations are appealing. The user can inquire on an impact on the subset of the one or more recommendations on reducing the risk score. In some implementations, the feedback can include a total amount that a user is willing to spend on lowering the risk score. In some implementations, the user may not have an infinite amount of disposable income, as such, the total amount that the user is willing to spend can be used as a variable in determining which recommendations the user should implement (e.g., to get the most bang for the buck).

The feedback can include user feedback that indicates that a certain recommendation is unnecessary (e.g., in the opinion of the user). In some implementations, a resident's injury may not be very severe to warrant certain recommendations, so the control system 290 can receive user feedback indicating that certain recommendations are not very beneficial. For example, a resident breaks his non-dominant hand and one recommendation is for the resident to install automated or voice-controlled lights and electronics (e.g., to aid the resident in controlling lights/electronics without having to use his non-dominant hand). User feedback in such an implementation can indicate that this recommendation is not very beneficial (e.g., in the opinion of the user) since the resident is able to comfortably use his dominant hand to control the lights/electronics, and such feedback can be received by and/or processed by the control system 290. In some implementations, the user can provide one or more reasons why a recommendation has not been implemented and/or why the recommendation is unnecessary or not beneficial (e.g., in the opinion of the user). The one or more reasons can be suggested by the control system 290, and the user can select a subset of reasons from the one or more reasons. An “other” category can be provided where the user can provide a reason different from any of the one or more reasons suggested by the control system 290.

In some implementations, where a portion of the recommendations were not implemented, for example, user feedback can include an updated status of the resident for use in providing an update risk score. For example, the resident may have multiple injuries, and as the resident heals or becomes better from his injuries, the resident becomes less prone to injuries and/or initially identified hazards become less hazardous for the resident. For example, a resident prescribed short-term use of a wheelchair due to fatigue can gradually get better over a period of time. Wheelchair use can fall from 10 hours per day to around 2 hours per day in only certain areas of the resident's living space. As such, user feedback providing more information on the improved health of the resident can be important to accurately identifying which hazards in the living space are still hazards to update the risk score. In some such implementations, if the health of the resident declines in one or more aspects, the user feedback on status can be used to identify one or more new hazards and/or determine an update risk score and/or determine one or more new recommendations.

At step 504, the control system 290 updates a risk score based at least in part on the feedback received at step 502. The control system 290 takes into account the additional information conveyed by the feedback to update the risk score according to some implementations of the present disclosure. For example, if the feedback is an implementation status indicating that at least one recommendation has been implemented, then the control system 290 can update a list of hazards and/or a likelihood of occurrence, taking into consideration the implementation status. The control system 290 can recalculate the risk score to reflect a current status of the living area after receiving feedback indicating one or more changes to the implementation status. Similarly, the risk score can be updated based on user feedback. As the resident's health improves and/or if a recommendation is minimally beneficial or non-beneficial, the control system 290 can recalculate the risk score accordingly.

In some implementations where the feedback includes an indication of which recommendations a resident or a user selects or finds appealing, the control system 290 can provide total estimated costs for following through with the selected recommendations. The control system 290 can provide an estimated impact on the risk score if the selected recommendations were undertaken. An advantage to providing the estimated impact on the risk score is to allow the user to simulate and/or experiment with various recommendations prior to committing resources to implementations. That way, the user can balance a recommendation's expected return on the risk score and cost of implementing the recommendation.

At step 506, the control system 290 provides one or more recommendations for reducing the updated risk score of step 504 to the user device 202. The control system 290 can provide the one or more recommendations in a similar manner as already described in connection to step 308 of FIG. 3 and/or step 408 of FIG. 4. In some implementations, the control system 290 can receive a total amount that the user is willing to spend on reducing the risk score at step 502. The control system 290 can then prioritize the one or more recommendations to determine a subset of the one or more recommendations that maximizes a reduction in the risk score while not exceeding the total amount that the user is willing to spend.

In some implementations, the control system 290 generates a report including the risk score prior to being updated, the one or more recommendations for reducing the risk score for the living space, the implementation status for each of the one or more recommendations for reducing the risk score, the updated risk score that is based at least in part on the risk score prior to being updated and the implementations status of each of the one or more recommendations, at least a portion of the resident profile, at least a portion of a virtual model of the living space, or any combination thereof. The control system 290 can cause the generated report to be sent to a caregiver, a family member of the resident, a friend of the resident, the resident, the user, or any combination thereof.

In some implementations, the control system 290 causes a reminder to be sent to the user device 202 and/or any other device. The reminder includes a listing of a subset of the one or more recommendations not implemented in the living space. The reminder can also show potential impacts to the risk score if at least a subset of the one or more recommendations were implemented. The reminder can include a push for implementing at least a subset of the one or more implementations.

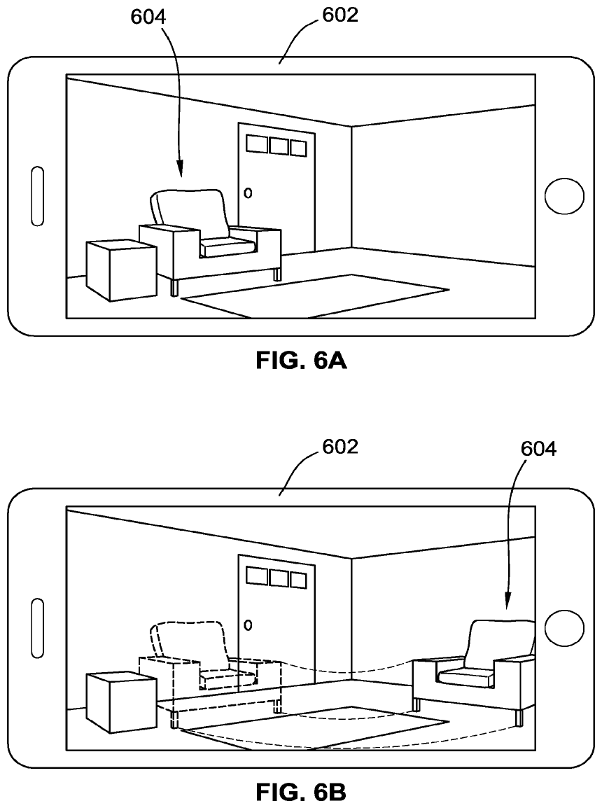

Referring to FIGS. 6A and 6B, a mobile device 602 (e.g., the same as, or similar to, the mobile device 104 in FIG. 1) operating according to some implementations of the present disclosure is illustrated. In FIG. 6A, the mobile device 602 depicts an image and/or virtual representation of a portion of a living space of a resident. The living space contains an armchair 604 at a first location. The displayed living space (in FIG. 6A) is how the living space appears to the user during an initial audit and/or review the living space.

Referring to FIG. 6B, the mobile device 602 now illustrates a first recommendation to move the armchair 604 to a second location. The first recommendation is based on the risk score analysis described herein. Implementation of the recommendation to move the armchair 604 is shown using an augmented reality view on the mobile device 602. As shown, the armchair 604 is being virtually animated and shown as being moved (in virtual reality, not reality) from the first location to the second location as depicted in FIG. 6B, thereby implementing the first recommendation (virtually). As such, the user is shown a simulation of what the first recommendation is and how the user can implement the first recommendation in reality.

In some implementations, software running on the mobile device 602 can recognize objects within the virtual representation of the portion of the living space. The mobile device 602 can identify that the armchair 604 is to be moved. The mobile device 602 can then generate the virtual representation without the object to be moved (e.g., the armchair 604). The mobile device 602 can determine a path of movement for the armchair 604. The mobile device 602 can then overlay the armchair 604 in the virtual representation showing the armchair 604 move along the path of movement.

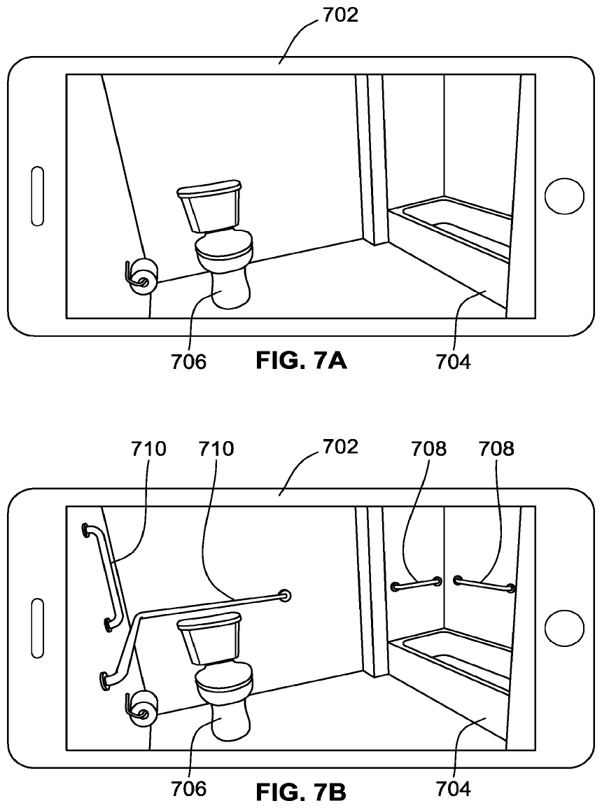

Referring to FIGS. 7A and 7B, a mobile device 702 (e.g., the same as, or similar to, the mobile device 104 in FIG. 1) operating according to some implementations of the present disclosure is illustrated. In FIG. 7A, the mobile device 702 depicts an image and/or a virtual representation of a portion of a bathroom/living space of a resident. The bathroom includes a bathtub 704 and a toilet 706. According to some implementations of the present disclosure, as described above, a recommendation for adding a number of grab bars into the bathroom of the resident is generated for the resident. The recommendation can be based on a profile of a resident, etc. In FIG. 7B, implementation of the recommendation is shown using an augmented reality view on the mobile device 702. As shown, the number of grab bars 708, 710 are virtually added near the bathtub 704 and near the toilet 706. That is, the grab bars 708 and 710 are virtual objects overlaid in the virtual representation of the bathroom even though physical grab bars are not present in the bathroom.

Some implementations of the present disclosure provide systems and/or methods for quantifying hazards and one or more recommendations for addressing the quantified hazards. The systems and/or methods can improve overall healthcare of a resident because as the one or more recommendations are being implemented, a doctor or caregiver can be made aware of the implementation progress. That way, a caregiver can follow up and provide additional feedback involving risks of re-hospitalization. Healthcare costs can be reduced because the system and method provide preventative measures to reduce hospitalization and/or re-hospitalization.

The methods and/or systems can allow for planning for getting a living space of a patient ready prior to discharging the patient from a care facility (e.g., a hospital). With recommendations being ranked and prioritized, caregivers or family members can determine which, if not all, of the ranked recommendations can be completed prior to the patient being discharged.

Some implementations of the present disclosure can be used in other contexts. For example, virtual objects can be placed in a virtual representation of a resident's home. The virtual objects can be linked to an online store for placing an order to purchase the virtual object. For example, when the resident needs to buy a new appliance, a virtual appliance can be generated and placed in a virtual representation of the resident's home. The virtual appliance can then serve as a link to a website for the resident to complete the purchase.

Described herein are systems and method for determining risk scores that are determined based at least in part on hazard scores associated with hazards in a living space of a resident. In some implementations, the risk scores and/or the underlying hazard score(s) can be improved by the system over time using machine learning (ML). By improved, it is meant that the risk scores and/or hazard scores are more accurate for a specific resident. For example, initially the systems of the present disclosure might set a hazard score for a rug in a room at 80 of 100, but over time a trained algorithm learns that the severity of a rug in a room is actually or more like 65 of 100. Further, the trained algorithm(s) can learn overtime how to weigh each hazard for the various different residents. For example, initially the systems of the present disclosure might set a weight for a hazard score for a resident recovering with a broken leg for the rug in a room at 90 of 100, but over time a trained algorithm learns that the weight for the hazard score for the resident recovering with the broken leg of a rug in a room is actually or more like 50 of 100. According to some such implementations, the systems of the present disclosure receive and/or collect data regarding various living spaces and various residents and analyze the data in view of actual injuries (e.g., falls, trips, slips, etc.) to learn how to better score specific hazards for specific types of residents and/or groups of residents.

While the present disclosure described systems and methods for determining risk scores and developing and/or implementing recommendations to reduce such risk scores, various aspects of the present disclosure can be used to aid a resident (e.g., a home owner, a realtor, etc.) in staging a home for sale or the like. That is, instead of making recommendations that make a room or living space safer or less hazardous for a resident, the recommendations of the present disclosure can be used to make a room or living space look relatively more spacious, relatively more aesthetically pleasing, relatively more updated, etc. In developing recommendations, instead of a hazards database, the system 200 can include a database of aesthetically pleasing living spaces, living spaces that look spacious, etc. In some implementations, the database can include features in living spaces that should be avoided (e.g., walls having multiple colors, relatively low lighting in the living space, etc.). The information in the database can be used, in a same or similar fashion as information in a hazards database, to propose changes/modifications to the resident's living space as described herein. The costs for making the proposed changed/modifications can also be included in the same, or similar fashion as described herein. As such, the user of the system can manipulate the outputs to get the most bang for the buck in terms of staging of the living space when trying to sell a home.

One or more elements or aspects or steps, or any portion(s) thereof, from one or more of any of claims 1-60 below can be combined with one or more elements or aspects or steps, or any portion(s) thereof, from one or more of any of the other claims 1-60 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations or alternative implementations according to aspects of the present disclosure may combine any number of features from any of the implementations described herein, such as, for example, in the alternative implementations described below.

1. A method including:
receiving data associated with a living space of a resident;
analyzing the data to identify one or more hazards in the living space;
determining a hazard score for each of the one or more identified hazards in the living space;
determining a likelihood of occurrence of harm for each of the one or more identified hazards in the living space; and
determining a risk score for the living space based at least in part on each of the respective hazard scores of the one or more identified hazards and each of the respective likelihoods of occurrence of harm for the one or more identified hazards, the risk score being indicative of how hazardous the living space is to the resident.

2-5. (canceled)

6. The method of claim 1, wherein each of the respective likelihoods of occurrence of harm is indicative of a percentage likelihood that the resident will encounter a respective one of the one or more hazards in the living space.

7. The method of claim 1, wherein the likelihood of occurrence for each respective one of the one or more hazards is based at least in part on a historical frequency of proximity of the resident to the respective one of the one or more hazards.

8. The method of claim 7, wherein a magnitude of the likelihood of occurrence for a respective one of the one or more hazards is directly proportional to the historical frequency of proximity.

9. The method of claim 1, wherein the determining the risk score for the living space further includes weighting the hazard score, for each of the one or more identified hazards, by the corresponding likelihood of occurrence and adding each of the weighted hazard scores to obtain the risk score.

10. The method of claim 1, wherein the one or more hazards include one or more obstacles blocking a walkway of the living space; a narrow walkway of the living space; one or more tripping hazards in the living space; one or more loose cords in the living space; one or more loose wires in the living space; a slippery floor of the living space; a relatively hard to reach light switch in the living space; a relatively hard to reach electrical outlet in the living space; a low toilet seat in the living space; a lack of shower handles in a shower in the living space; a sharp corner in the living space; a sharp edge in the living space; one or more pets in the living space; one or more stairs in the living space; a gas stove in the living space; a high positioning of cabinets in the living space; a difficult-to-use electrical appliance in the living space; a height of a bed in the living space, a height of a sink in a bathroom of the living space, a dimly lit area in the living space, a type of shower or tub of the living space, or any combination thereof.

11. The method of claim 1, wherein the received data is received from a sensor including one or more cameras, one or more infrared cameras, one or more acoustic sensors, one or more radar sensors, one or more ultra-wide band RF sensors, one or more microphones, one or more speakers, one or more heat sensors, one or more gas sensors, one or more barometers, one or more proximity sensors, one or more humidity sensors, one or more gyroscopic sensors, one or more light sensors, one or more motion sensors, or any combination thereof.

12. The method of claim 1, further comprising:
generating one or more recommendations for reducing the risk score for the living space, wherein the generated one or more recommendations for reducing the risk score for the living space include increasing a width of a physical walkway of the living space, removing a first physical object from the living space, reorienting a second physical object within the living space, adding one or more new objects to the living space, or any combination thereof.

13-16. (canceled)

17. The method of claim 12, further comprising:
creating, using at least a portion of the received data, a virtual representation of the living space, wherein the virtual representation of the living space includes one or more virtual items of furniture, one or more virtual rugs, one or more virtual carpeted floor areas, one or more virtual light fixtures, one or more virtual hardwood floor areas, one or more virtual electrical outlets, one or more virtual stairs, one or more virtual appliances, one or more virtual cabinets, one or more virtual items of clothing, one or more virtual shoes, a virtual bed, one or more virtual pets, one or more virtual doorways, one or more virtual walkways, or any combination thereof.

18. (canceled)

19. The method of claim 17, further comprising:
causing a representation of at least one of the generated one or more recommendations to be displayed as one or more virtual objects within the virtual representation of the living space.

20-21. (canceled)

22. The method of claim 19, wherein at least one of the one or more virtual objects is a moving virtual object, the moving virtual object including (i) an item of furniture moving from one location to another, (ii) a rug moving from one location to another, (iii) the virtual object moving from one location to another, or (iv) any combination thereof.

23. (canceled)

24. The method of claim 19, wherein the one or more virtual objects are displayed within the virtual representation of the living space in an augmented reality view of at least a portion of the living space, the augmented reality view being presented via an electronic display device.

25. The method of claim 17, further comprising:
causing a representation of at least one of the generated one or more recommendations to be displayed as one or more animations within the virtual representation of the living space.

26. The method of claim 25, wherein the one or more animations includes (i) a widening of a virtual walkway in the virtual representation of the living space, (ii) a narrowing of the virtual walkway in the virtual representation of the living space, (iii) a resizing of a virtual object in the virtual representation of the living space, (iv) a widening of a virtual doorway in the virtual representation of the living space, (v) a narrowing of the virtual doorway in the virtual representation of the living space, or (iv) any combination thereof.

27-31. (canceled)

32. The method of claim 12, further comprising:
causing the one or more recommendations to be sent to an electronic device;
receiving implementation feedback via the electronic device, the implementation feedback associated with an implementation status for each of the one or more recommendations for reducing the risk score; and
generating a report, the report including the risk score, the one or more recommendations for reducing the risk score for the living space, the implementation status for each of the one or more recommendations for reducing the risk score, a revised risk score that is based at least in part on the risk score and the implementations status of each of the one or more recommendations, at least a portion of the resident profile, at least a portion of a virtual model of the living space, or any combination thereof.

33-34. (canceled)

35. The method of claim 1, wherein the resident profile includes an electronic health record associated with the resident, a height of the resident, an age of the resident, a health condition of the resident, a rooming status of the resident, a type of home of the resident, medication prescribed to the resident, vital signs of the resident, a type of assistive device used by the resident, a mental status of the resident, demographic information of the resident, a location of a bedroom of the resident in the living space, a location of a bathroom of the resident in the living space, an average number of times the resident uses the bathroom per day in the living space, an average activity level of the resident per day, or any combination thereof.

36. (canceled)

37. The method of claim 1, further comprising:
creating, using at least a portion of the received data, a virtual representation of the living space, wherein the virtual representation of the living space includes (i) a virtual walkway, (ii) a virtual object, or (iii) both (i) and (ii), the virtual walkway being a virtual representation of a physical walkway contained in the living space and the virtual object being a virtual representation of a physical object contained in the living space.

38. The method of claim 37, further comprising:
determining (i) a width of the physical walkway, (ii) a distance between the physical object and a second physical object in the living space, (iii) a size of the physical object, (iv) one or more dimensions of the physical object, or (v) any combination thereof.

39-44. (canceled)

45. A system comprising:
a sensor configured to generate data associated with a living space of a resident;
a memory storing machine-readable instructions and a resident profile; and
a control system including one or more processors configured to execute the machine-readable instructions to:
analyze the data to identify one or more hazards in the living space;
determine a hazard score for each of the one or more identified hazards in the living space;
determine a likelihood of occurrence of harm for each of the one or more identified hazards in the living space; and
determine a risk score for the living space based at least in part on each of the respective hazard scores of the one or more identified hazards and each of the respective likelihoods of occurrence of harm for the one or more identified hazards, the risk score being indicative of how hazardous the living space is to the resident.

46-49. (canceled)

50. The system of claim 45, wherein the control system is further configured to execute the machine-readable instructions to generate one or more recommendations for reducing the risk score for the living space, wherein the generated one or more recommendations for reducing the risk score for the living space include a first recommendation to remove a first physical object from the living space, a second recommendation to reorient a second physical object within the living space, a third recommendation to add one or more new objects to the living space, or any combination thereof.

51-52. (canceled)

53. The system of any one of claim 45, wherein the control system is further configured to execute the machine-readable instructions to create, using at least a portion of the generated data, a virtual representation of the living space, the virtual representation of the living space including one or more virtual items of furniture, one or more virtual rugs, one or more virtual carpeted floor areas, one or more virtual light fixtures, one or more virtual hardwood floor areas, one or more virtual electrical outlets, one or more virtual stairs, one or more virtual appliances, one or more virtual cabinets, one or more virtual items of clothing, one or more virtual shoes, a virtual bed, one or more virtual pets, one or more virtual doorways, one or more virtual walkways, or any combination thereof.

54. The system of claim 53, wherein the control system is further configured to execute the machine-readable instructions to cause a representation of at least one of the generated one or more recommendations to be displayed as one or more virtual objects within the virtual representation of the living space.

55. (canceled)

56. The system of claim 54, wherein the control system is further configured to execute the machine-readable instructions to cause a representation of at least one of the generated one or more recommendations to be displayed as one or more animations within the virtual representation of the living space, the one or more animations including (i) a widening of a virtual walkway in the virtual representation of the living space, (ii) a narrowing of the virtual walkway in the virtual representation of the living space, (iii) a resizing of a virtual object in the virtual representation of the living space, (iv) a widening of a virtual doorway in the virtual representation of the living space, (v) a narrowing of the virtual doorway in the virtual representation of the living space, or (iv) any combination thereof.

57-60. (canceled)
